(19) United States
(12) Patent Application Publication
(10) Pub. No.: US 2013/0281551 A1
(43) Pub. Date: Oct. 24, 2013

(54) Methods Relating to Personal Care Compositions

(71) Applicant: THE PROCTER & GAMBLE COMPANY, Cincinnati, OH (US)

(72) Inventors: Qing STELLA, Cincinnati, OH (US); Gerald John GUSKEY, Symmes Twp., OH (US); Cynthia Ann GARZA, Cincinnati, OH (US); Timothy Woodrow COFFINDAFFER, Maineville, OH (US); John David CARTER, Mason, OH (US); Kenneth Eugene KYTE, III, Oregonia, OH (US)

(73) Assignee: The Procter & Gamble Company, Cincinnati, OH (US)

(21) Appl. No.: 13/865,588

(22) Filed: Apr. 18, 2013

Related U.S. Application Data
(60) Provisional application No. 61/635,884, filed on Apr. 20, 2012.

(51) Int. Cl. A61K 8/37; A61Q 19/00

(52) U.S. Cl. CPC A61K 8/37; A61Q 19/00
USPC 514/785

(57) ABSTRACT

A personal cleansing composition includes a cleansing phase and a benefit phase, the benefit phase having a hydrophobic benefit agent and one or more oligomers derived from metathesis of unsaturated polyol esters and methods relating thereto.

### FIELD OF THE INVENTION

The present disclosure generally relates to a rinse-off personal cleansing composition with a benefit phase having a benefit agent and one or more oligomers derived from metathesis of unsaturated polyol esters and methods pertaining thereto.

### BACKGROUND OF THE INVENTION

Cleansing the skin is an activity that has been done for millennia. Skin cleansing and methods therefore have involved the utilization of soaps, body washes, and other personal cleansing compositions. Personal cleansing compositions can be structured to suspend and stabilize dispersions of benefit agents while maintaining physical integrity of the compositions. The ability to deposit benefit agents and hydrate the skin while maintaining physical integrity can be an important benefit for such compositions. Oils, for example, are a type of benefit agent for skin hydration improvement. However, it is known that many such benefit agents can exhibit strong interactions with surfactants which can cause product instability and low deposition. Achieving a proper balance between stability in a composition and performance properties such as increased deposition and enhanced skin hydration can be a difficult task, and as such, it is desirable to provide a personal cleansing composition to effectively improve deposition of benefit agents and enhance skin hydration.

### SUMMARY OF THE INVENTION

A method of enhancing deposition of a hydrophobic benefit agent on skin, comprising: providing a rinse-off personal cleansing composition comprising a cleansing phase comprising a surfactant and water and a benefit phase comprising a benefit agent and from about 1% to about 15% of an oligomer derived from metathesis of unsaturated polyol esters, and instructing an individual to apply the composition to the skin.

A method of enhancing the hydration of skin, comprising: providing a rinse-off personal cleansing composition comprising a cleansing phase comprising a surfactant and water and a benefit phase comprising a benefit agent and from about 1% to about 15% of an oligomer derived from metathesis of unsaturated polyol esters, and instructing an individual to apply the composition to the skin, wherein the level of hydration is in accordance with at least one of the following: a Corneometer improvement of 0.1 Corneometer units or more as compared to the personal cleansing composition without the oligomer after treatment of at least once a day for at least 3 days when measured in accordance with the Corneometer Testing Method; a dry skin grade improvement of about 0.4 units or more at 3 hours after a treatment versus the personal cleansing composition without the oligomer when measured in accordance with the Dry Skin Grade Method; a TEWL improvement of about 0.5 grams of water per hour per m² or more after treatment at least once a day for 14 days when measured in accordance with the TEWL Method, or a combination thereof.

### DETAILED DESCRIPTION OF THE INVENTION

This application incorporates by reference in its entirety U.S. Provisional App. No. 61/635,884 filed Apr. 20, 2012, to which this application claims priority.

While the specification concludes with the claims particularly pointing and distinctly claiming the invention, it is believed that the present invention will be better understood from the following description.

The devices, apparatuses, methods, components, and/or compositions of the present invention can include, consist essentially of, or consist of, the components of the present invention as well as other ingredients described herein. As used herein, “consisting essentially of” means that the devices, apparatuses, methods, components, and/or compositions may include additional ingredients, but only if the additional ingredients do not materially alter the basic and novel characteristics of the claimed devices, apparatuses, methods, components, and/or compositions.

All measurements used herein are in metric units unless otherwise specified.

### I. DEFINITIONS

As used herein, the following terms shall have the meaning specified thereafter:

“Anhydrous” refers to those compositions, and components thereof, which are substantially free of water.

“Associative polymer” refers to a water-dispersible polymer comprising hydrophobic groups at an end or pendants to a hydrophilic backbone.

“Dry skin” refers to a term used by consumers, cosmetic scientists, and dermatologists. Dry skin can be characterized by a rough, scaly, and/or flaky skin surface, especially in low humidity conditions and is often associated with the somatory sensations of tightness, itch, and/or pain.

“Multiphase” refers to compositions comprising at least two phases which can be chemically distinct (e.g., a structured cleansing phase and a benefit phase). Such phases can be in direct physical contact with one another. For example, a personal cleansing composition can be a multiphase personal cleansing composition where phases of the personal cleansing composition can be blended or mixed to a significant degree, but still be physically distinct. In these situations, the physical distinctiveness is undetectable to the naked eye. As another example, the personal cleansing composition can be a multiphase personal cleansing composition where phases of the personal cleansing composition can be made to occupy distinct physical spaces inside a package in which the phases can be stored. In these situations, the phases are in physical contact to some degree and are visually distinct. Visually distinct phases can take many forms (e.g., phases can appear as striped, marbled).

“Non-associative polymer” refers to a water-dispersible polymer with a relatively uniform hydrophilic backbone lacking hydrophobic groups.

“Non-diseased skin” refers to skin that is generally free of disease, infection, and/or fungus. As used herein, dry skin is considered to be included in non-diseased skin.

“Personal cleansing composition” refers to compositions intended for topical application to skin. The personal cleansing compositions can be extrudable or dispensable from a package. The personal cleansing compositions can be in the form of, for example, a liquid, semi-liquid cream, lotion, or gel and are intended for topical application to the skin. Examples of personal cleansing compositions can include but are not limited to bar soap, body wash, moisturizing body wash, shower gels, skin cleansers, cleansing milks, in shower body moisturizer, shaving preparations, and cleansing compositions used in conjunction with a disposable cleansing cloth.

“Rinse-off” refers to personal cleansing compositions that are designed to be rinsed from the skin within seconds to minutes of application. The product could also be wiped off using a substrate.

“STnS” refers to sodium trideceth(n) sulfate, wherein n can define the average number of moles of ethoxylate per molecule.

“Structured” refers to having a rheology that can confer stability on the personal cleansing composition. A degree of structure can be determined by characteristics determined by a Zero Shear Viscosity Method described in U.S. Pub. No. 2012/0009285 by Wei et al. Accordingly, a structured cleansing phase of the personal cleansing composition can be considered to be structured if the structured cleansing phase has a Zero Shear Viscosity of about 20 Pascal-seconds (Pa-s) or more, about 200 Pa-s or more, about 500 Pa-s or more, about 1,000 Pa-s or more, about 1,500 Pa-s or more, or about 2,000 Pa-s or more. Other methods for determining characteristics which can define a degree of structure are also described in U.S. Pub. No. 2012/0009285.

The phrase “substantially free of” as used herein, unless otherwise specified means that the personal cleansing composition comprises less than about 3% of the stated ingredient. Further the composition could contain less than about 1% or even less than about 0.1% of the stated ingredient. The term “free of” as used herein means that the personal cleansing composition comprises 0% of the stated ingredient, that is the ingredient has not been added to the personal cleansing composition. However, these ingredients may incidentally form as a byproduct or a reaction product of the other components of the personal cleansing composition.

### II. PERSONAL CLEANSING COMPOSITIONS

A rinse-off personal cleansing composition can be a multiphase composition. Such multiphase compositions can include at least two phases which can be chemically distinct. For example, personal cleansing compositions can include a cleansing phase and a benefit phase. The benefit phase can include one or more benefit agents that can be deposited on the skin of an individual to provide improved appearance, increased skin hydration, and other desired benefits. There are several benefit agents that can provide such desired benefits. However, providing sufficient deposition of such benefit agents and achieving a desired level of skin hydration from a rinse-off can be a difficult task. As a result, there is a continued interest in improving deposition of benefit agents and enhancing skin hydration from a rinse-off.

Benefit agents, by themselves, can lack certain properties that promote deposition from a rinse-off. Some benefit agents may not possess a desired viscosity or structure to provide sufficient adhesion to skin For example, oils (e.g., soybean oil) can be too liquid-like and can lack a necessary visco-elasticity to provide sufficient deposition. Additionally, some benefit agents may not provide sufficient particle sizes to allow for adequate deposition. Though particle size can be increased with a coacervate, such benefit agents can reduce the rheological modulus of the coacervate such that there is a less significant increase in deposition than would be expected with the use of a coacervate.

Without wishing to be bound by theory, it is believed that using one or more oligomers derived from metathesis of unsaturated polyol esters, like a soy oligomer, in the benefit phase, along with a benefit agent (e.g., hydrophobic benefit agent), can improve deposition of the benefit agent and/or enhance skin hydration. It is believed that polymeric characteristics of such oligomers can result in a higher efficiency of rheology modification and oil compatibility than that resulting from hydrogenated waxes or from hydrogenating the oils. Hydrogenation of the oils is sometimes used to increase the viscosity of the oils.

Rheology of a polymer in a solvent can depend on molecular size and concentration, and such oligomer molecules can form much larger and extended conformations than hydrogenated waxes and oils. Thus, using such oligomers in the benefit phase can promote overlapping of oligomer molecules for network formation and can modify oil rheology. In particular, it is believed that such oligomers, like soy oligomers, can combine with a hydrophobic benefit agent to form a more visco-elastic benefit phase and larger particles, which are conducive for increased deposition. Such improved deposition of a benefit agent is illustrated by the examples in Tables 2 (comparative) and 3 inventive, provided below. For example, the deposition of benefit agent in Comparative Example 1 was 13 μg/cm² versus Inventive Example 8 which was 731 μg/cm² and the only difference in the two is the substitution of 10% of the benefit agent with a soy oligomer in Example 8.

Further, and as shown in additional examples herein, inclusion of an oligomer, like soy oligomer, within the benefit phase can also allow for enhanced skin hydration. In particular, it is believed that the inclusion of the oligomer within the benefit phase can enhance skin hydration by increasing occlusivity in a benefit agent to prevent water loss from the skin and provide a higher benefit phase viscosity so as to weigh down skin flakes, resulting in lower dry skin grade. Some exemplary dry skin grade results are shown in Table 9, below, where after three days of treatment, Inventive Example 11 showed better dry skin grade relative to Comparative Example 7, and thus effected a greater hydration level. Additional dry skin grade measurements are shown in Table 10, where the measurements are taken at 24 hours after the last treatment, and Inventive Example 11 shows better dry skin grade than Comparative Example 7 at all measured points.

Better skin hydration is also exemplified in a reduction in the Transepidermal Water Loss (TEWL), as seen in Tables 5 and 6. Table 5 shows the TEWL measurements at 3 hours after the last treatment on days 0, 3, 5, 14, and 21. While the TEWL measurements are similar at day 0, there is a noticeable difference at days 14 and 21 with the oligomer containing Inventive Example 11 having better TEWL than the comparative example. The same is true for Table 6 which it is looking at the same compositions, but 24 hours after the last treatment.

Another way of looking at skin hydration is with a corneometer. The higher the number, the better the hydration. Looking at Tables 7 and 8, below, Inventive Example 11 showed higher Corneometer values at all measured times than Comparative Example 7, showing Inventive Example 11 provided better hydration of the skin.

It is further believed that using such oligomers, like a soy oligomer, in the benefit phase can allow a personal cleansing composition to exhibit a crossover stress value that can be conducive for improved delivery and retention of a benefit agent on the skin of an individual, especially during rinse off. It is believed that using materials with a high crossover stress value can result in poor delivery. However, it is believed that materials with a low crossover stress value can behave like liquids, which can result in improved delivery, but poor retention. Thus, to provide adequate delivery and retention, it is desirable for the benefit phase to exhibit a crossover stress that is not too high or too low. Thus, the benefit phase of a personal cleansing composition can exhibit a crossover stress, fro example, in a range of from about 20 Pa to about 200 Pa; from about 50 Pa to about 190 Pa; from about 80 Pa to about 180 Pa; from about 90 Pa to about 170 Pa, or any combination thereof. A method for determining the crossover stress value for the benefit phase of a composition is described below in the Benefit Phase Rheology Method.

A. Cleansing Phase

As noted herein, a personal cleansing composition can be a multi-phase composition and can include a cleansing phase and a benefit phase. The cleansing phase can be a structured cleansing phase. The cleansing phase and benefit phase can be in physical contact.

A personal cleansing composition can comprise, for example, from about 0.1% to 25%, from about 0.5% to about 20%, or from about 1.0% to about 15%, by weight of a the personal cleansing composition, of a surfactant or a cosurfactant. Surfactants can comprise, for example, anionic surfactants, soaps, interrupted soaps, detergents, non-ionic surfactants, amphoteric surfactants, zwitterionic surfactants, or mixtures thereof. For instance, the personal cleansing composition can include an amphoteric surfactant and/or a zwitterionic surfactant. Suitable amphoteric or zwitterionic surfactants can include those described in U.S. Pat. Nos. 5,104,646 and 5,106,609.

Soaps may include, for example, the sodium, potassium and lower alkanolamine (preferably triethanolamine) salts of C12 to 22, preferably C14 to 18, fatty acids. Typical fatty acids include lauric, myristic, palmitic and stearic acid and mixtures thereof. The preferred fatty acids are palmitic and stearic. The soaps may be utilized in the preneutralized form (i.e., as the sodium, potassium or alkanolamine salt) or in the free acid form followed by subsequent neutralization with sodium hydroxide, potassium hydroxide and/or lower alkanolamine (preferably triethanolamine). In any event, the final composition preferably contains sufficient base to neutralize or partially neutralize the soap component and adjust the pH to the desired level (typically between 5 and 10, more typically between 6 and 9).

A cleansing phase can include from about 1% to about 20%, from about 2% to about 15%, from about 5% to about 10%, or any combination thereof, by weight of the personal cleansing composition of STnS, wherein n can define average moles of ethoxylation. n can range from about 0 to about 3, from about 0.5 to about 2.7, from about 1.1 to about 2.5, from about 1.8 to about 2.2, or n can be about 2. When n is less than 3, STnS can provide improved stability, improved compatibility of benefit agents within the personal cleansing compositions, and increased mildness of the personal cleansing compositions, such described benefits of STnS are disclosed in U.S. Patent Pub. No. 2012/0009285.

Amphoteric surfactants can include those that can be broadly described as derivatives of aliphatic secondary and tertiary amines in which an aliphatic radical can be straight or branched chain and wherein an aliphatic substituent can contain from about 8 to about 18 carbon atoms such that one carbon atom can contain an anionic water solubilizing group, e.g., carboxy, sulfonate, sulfate, phosphate, or phosphonate. Examples of compounds falling within this definition can be sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and products described in U.S. Pat. No. 2,528,378. Other examples of amphoteric surfactants can include sodium lauroamphoacetate, sodium cocoamphoactetate, disodium lauroamphoacetate disodium cocodiamphoacetate, and mixtures thereof. Amphoacetates and diamphoacetates can also be used.

Zwitterionic surfactants suitable for use can include those that are broadly described as derivatives of aliphatic quaternary ammonium, phosphonium, and sulfonium compounds, in which aliphatic radicals can be straight or branched chains, and wherein an aliphatic substituent can contain from about 8 to about 18 carbon atoms such that one carbon atom can contain an anionic group, e.g., carboxy, sulfonate, sulfate, phosphate, or phosphonate. Other zwitterionic surfactants can include betaines, including cocoamidopropyl betaine.

A cleansing phase can also include an associative and/or non-associative polymer. These polymers can help provide structure to the phase. Associative polymers used in the cleansing phase can be a crosslinked, alkali swellable, associative polymer comprising acidic monomers and associative monomers with hydrophobic end groups, whereby the associative polymer comprises a percentage hydrophobic modification and a hydrophobic side chain comprising alkyl functional groups. Without intending to be limited by theory, it is believed the acidic monomers can contribute to an ability of the associative polymer to swell in water upon neutralization of acidic groups; and associative monomers anchor the associative polymer into surfactant hydrophobic domains, e.g., lamellae, to confer structure to the surfactant phase and keep the associative polymer from collapsing and losing effectiveness in a presence of an electrolyte. The crosslinked, associative polymer can comprise a percentage hydrophobic modification, which is a mole percentage of monomers expressed as a percentage of a total number of all monomers in a polymer backbone, including both acidic and other non-acidic monomers. Percentage hydrophobic modification of the associative polymer, hereafter % HM, can be determined by the ratio of monomers added during synthesis, or by analytical techniques such as proton nuclear magnetic resonance (NMR). Associative alkyl side chains can comprise, for example, butyl, propyl, stearyl, steareth, cetyl, lauryl, laureth, octyl, behenyl, beheneth, steareth, or other linear, branched, saturated, or unsaturated alkyl or alketh hydrocarbon side chains.

One exemplary associative polymer is AQUPEC® SER-300 made by Sumitomo Seika of Japan, which is an acrylate/C₁₀-C₃₀ alkyl acrylate cross-polymer and comprises stearyl side chains with less than about 1% HM. Associative polymers can comprise about C₁₆ (cetyl) alkyl hydrophobic side chains with about 0.7% hydrophobic modification, but a percentage hydrophobic modification can be up to an aqueous solubility limit in surfactant compositions (e.g., up to 2%, 5%, or 10%). Other associative polymers can include stearyl, octyl, decyl and lauryl side chains, alkyl acrylate polymers, polyacrylates, hydrophobically-modified polysaccharides, hydrophobically-modified urethanes, AQUPEC® SER-150 (acrylate/C₁₀-C₃₀ alkyl acrylate cross-polymer) comprising about C₁₈ (stearyl) side chains and about 0.4% HM, and AQUPEC® HV-701EDR which comprises about C₈ (octyl) side chains and about 3.5% HM, and mixtures thereof. An additional exemplary associative polymer is Stabylen 30 manufactured by 3V Sigma S.p.A., which has branched isodecanoate hydrophobic associative side chains.

The cleansing phase of a personal cleansing composition can further include a non-associative polymer. Suitable non-associative polymers can include water-dispersible polymers with relatively uniform hydrophilic backbone lacking hydrophobic groups. Examples of non-associative polymers can include biopolymer polysaccharides (e.g., xanthan gum, gellan gum), cellulosic polysaccharides (e.g., carboxymethyl cellulose, carboxymethyl hydroxyethyl cellulose), other polysaccharides (e.g., guar gum, hydroxypropyl guar, and sodium alginate), synthetic hydrocarbon polymers (e.g., polyacrylamide and copolymers, polyethylene oxide, polyacrylic acid copolymers), or combinations thereof.

Personal cleansing compositions can additionally comprise an organic cationic deposition polymer in one or more phases as a deposition aid for benefit agents described herein. Suitable cationic deposition polymers can contain cationic nitrogen-containing moieties such as quaternary moieties. Non-limiting examples of cationic deposition polymers can include polysaccharide polymers, such as cationic cellulose derivatives. Cationic cellulose polymers can be salts of hydroxyethyl cellulose reacted with trimethyl ammonium substituted epoxide, referred to in the industry (CTFA) as Polyquaternium 10, which can be available from Amerchol Corp. (Edison, N.J.) in their Polymer KG, JR, and LR series of polymers. Other suitable cationic deposition polymers can include cationic guar gum derivatives, such as guar hydroxypropyltrimonium chloride, specific examples of which can include the Jaguar series commercially available from Rhodia Inc. and N-Hance polymer series commercially available from Aqualon. Suitable water-soluble cationic deposition polymers can include synthetic polyacrylamides such as Polyquaternium 76 and Polymethylene-bis-acrylamide methacrylamido propyltrimethyl ammonium chloride (PAM/MAPTAC). Such PAM/MAPTAC can have an acrylamide to methacrylamido propyltrimethyl ammonium chloride ratio of 88:12. The deposition polymers can have a cationic charge density from about 0.8 meq/g to about 2.0 meq/g or from about 1.0 meq/g to about 1.5 meq/g, for example.

A cleansing phase of a personal cleansing composition can also include water. The cleansing phase can comprise from about 10% to about 90%, from about 40% to about 85%, or from about 60% to about 80%, by weight of the cleansing phase, of water.

Other optional additives can be included in the cleaning phase, including for example an emulsifier (e.g., non-ionic emulsifier) and electrolyes. Suitable electrolytes can includes an anion such as phosphate, chloride, sulfate, citrate, and mixtures thereof and a cation such as sodium, ammonium, potassium, magnesium, and mixtures thereof. For example, suitable electrolytes can include sodium chloride, ammonium chloride, sodium sulfate, ammonium sulfate, and mixtures thereof. Other suitable emulsifiers and electrolytes are described in U.S. Patent Pub. No. 2012/0009285.

B. Benefit Phase

As noted herein, personal cleansing compositions can include a benefit phase. The benefit phase can be hydrophobic and/or anhydrous. The benefit phase can also be substantially free of or free of surfactant.

The benefit phase can include one or more benefit agents. In particular, the benefit phase can comprise from about 0.1% to about 50%, by weight of the personal cleansing composition, of a benefit agent. The benefit phase can include, for example, from about 0.5% to about 20% or from about 1.0% to about 10%, by weight of the personal cleansing composition, of the benefit agent. Such benefit agents can include water insoluble agents or hydrophobic benefit agents.

Non-limiting examples of benefit agents include petrolatum, glyceryl monooleate, mineral oil, natural oils (e.g., soybean oil, saturated or unsaturated), sucrose esters, cholesterol, fatty esters, fatty alcohols, and mixtures thereof. Other suitable benefit agents are described in U.S. Patent Pub. No. 2012/0009285.

Additional non-limiting examples of benefit agents include SEFOSE®, lanolin esters, lanolin oil, natural waxes, synthetic waxes, volatile organosiloxanes, derivatives of volatile organosiloxanes, non-volatile organosiloxanes, derivatives of non-volatile organosiloxanes, natural triglycerides, synthetic triglycerides, and combinations thereof.

SEFOSE® includes one or more types of sucrose polyesters. Sucrose polyesters are derived from a natural resource and therefore, the use of sucrose polyesters as the benefit agents can result in a positive environmental impact. Sucrose polyesters are polyester materials, having multiple substitution positions around the sucrose backbone coupled with the chain length, saturation, and derivation variables of the fatty chains. Such sucrose polyesters can have an esterification (“IBAR”) of greater than about 5. The sucrose polyester may have an IBAR of from about 5 to about 8; of about 5-7; of about 6; or of about 8. As sucrose polyesters are derived from a natural resource, a distribution in the IBAR and chain length may exist. For example a sucrose polyester having an IBAR of 6, may contain a mixture of mostly IBAR of about 6, with some IBAR of about 5 and some IBAR of about 7. Additionally, such sucrose polyesters may have a saturation or iodine value (“IV”) of about 3 to about 140. The sucrose polyester may have, for example, an IV of about 10 to about 120 or of about 20 to 100. Further, such sucrose polyesters can have a chain length of about C₁₂ to C₂₀.

Non-limiting examples of sucrose polyesters suitable for use include SEFOSE® 1618S, SEFOSE® 1618U, SEFOSE® 1618H, Sefa Soyate IMF 40, Sefa Soyate LP426, SEFOSE® 2275, SEFOSE® C1695, SEFOSE® C18:0 95, SEFOSE® C1495, SEFOSE® 1618H B6, SEFOSE® 1618S B6, SEFOSE® 1618U B6, Sefa Cottonate, SEFOSE® C1295, Sefa C895, Sefa C1095, SEFOSE® 1618S B4.5, all available from The Procter and Gamble Co. of Cincinnati, Ohio.

Non-limiting examples of glycerides suitable for use as hydrophobic skin benefit agents herein can include castor oil, safflower oil, corn oil, walnut oil, peanut oil, olive oil, cod liver oil, almond oil, avocado oil, palm oil, sesame oil, soybean oil, unsaturated soybean oil, vegetable oils, sunflower seed oil, vegetable oil derivatives, coconut oil and derivatized coconut oil, cottonseed oil and derivatized cottonseed oil, jojoba oil, cocoa butter, and combinations thereof.

Non-limiting examples of silicone oils suitable for use as hydrophobic skin benefit agents herein can include dimethicone copolyol, dimethylpolysiloxane, diethylpolysiloxane, mixed C₁-C₃₀ alkyl polysiloxanes, phenyl dimethicone, dimethiconol, and combinations thereof. Non-limiting examples of silicone oils useful herein are described in U.S. Pat. No. 5,011,681. Still other suitable hydrophobic skin benefit agents can include milk triglycerides (e.g., hydroxylated milk glyceride) and polyol fatty acid polyesters.

A hydrophobic benefit agent can exhibit a Vaughan solubility parameter from about 5 to about 14 and exhibit a viscosity of about 1500 cP or less at from about 20° C. to about 25° C. Vaughan solubility parameters are defined in Vaughan in Cosmetics and Toiletries, Vol. 103. Non-limiting examples of hydrophobic materials having Vaughan solubility parameter values in the above range can include the following: Cyclomethicone, 5.92; Squalene, 6.03; Petrolatum, 7.33; Isopropyl Palmitate, 7.78; Isopropyl Myristate, 8.02; Castor Oil, 8.90; Cholesterol, 9.55; as reported in Solubility, Effects in Product, Package, Penetration and Preservation, C. D. Vaughan, Cosmetics and Toiletries, Vol. 103, October 1988.

The benefit agents can be combined with one or more oligomers derived from metathesis of unsaturated polyol esters. The benefit phase can include, for example, from about 1% to about 15%, from about 1% to about 13%, from about 1% to about 11%, from about 1% to about 10%; or from about 2% to about 10%, by weight of the benefit phase, of the oligomer.

Examples of such oligomers and methods for making them may be found in U.S. Patent Application Publication no. 2009/0220443, entitled “Compositions Comprising Unsaturated Polyol Esters” by Braksmayer et al., incorporated herein by reference. The oligomers may be self metathesized or cross-metathesized, for example. The oligomer may be a triglyceride oligomer. The oligomer may be a soy oligomer. The oligomers range from partially hydrogenated to fully hydrogenated. The oligomers can also be branched containing oligomers.

A metathesized unsaturated polyol ester refers to the product obtained when one or more unsaturated polyol ester ingredient(s) are subjected to a metathesis reaction. Metathesis is a catalytic reaction that involves the interchange of alkylidene units among compounds containing one or more double bonds (i.e., olefinic compounds) via the formation and cleavage of the carbon-carbon double bonds. Metathesis may occur between two of the same molecules (often referred to as self-metathesis) and/or it may occur between two different molecules (often referred to as cross-metathesis). Self-metathesis may be represented schematically as shown in Equation I:

R¹—CH═CH—R²+R¹—CH═CH—R² [symbol] R¹—CH═CH—R¹+R²CH═CH—R² (I)

where R¹ and R² are organic groups.

Cross-metathesis may be represented schematically as shown in Equation II:

R¹—CH═CH—R²+R³—CH═CH—R⁴ [symbol] R¹—CH═CH—R³+R—CH═CH—R⁴+R²—CH═CH—R³+R²—CH═CH—R⁴+R¹—CH═CH—R¹+R²—CH═CH—R²+R³—CH═CH—R³+R⁴—CH═CH—R⁴ (II)

where R¹, R², R³, and R⁴ are organic groups.

When the unsaturated polyol ester comprises molecules that have more than one carbon-carbon double bond (i.e., a polyunsaturated polyol ester), self-metathesis results in oligomerization of the unsaturated polyol ester. The self-metathesis reaction results in the formation of metathesis dimers, metathesis trimers, and metathesis tetramers. Higher order metathesis oligomers, such as metathesis pentamers and metathesis hexamers, may also be formed by continued self-metathesis and will depend on the number and type of chains connecting the unsaturated polyol ester material as well as the number of esters and orientation of the ester relative to the unsaturation.

As a starting material, metathesized unsaturated polyol esters are prepared from one or more unsaturated polyol esters. As used herein, the term “unsaturated polyol ester” refers to a compound having two or more hydroxyl groups wherein at least one of the hydroxyl groups is in the form of an ester and wherein the ester has an organic group including at least one carbon-carbon double bond. An exemplary unsaturated polyol ester can be represented by the general structure I:

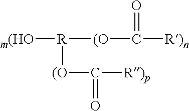

where n≧1; m≧0; p≧0; (n+m+p)≧2; R is an organic group; R′ is an organic group having at least one carbon-carbon double bond; and R″ is a saturated organic group. Examples of the unsaturated polyol ester are described in detail in U.S. 2009/0220443 A1.

The unsaturated polyol ester, for example, is an unsaturated ester of glycerol. Sources of unsaturated polyol esters of glycerol include synthesized oils, natural oils (e.g., vegetable oils, algae oils, bacterial derived oils, and animal fats), combinations of theses, and the like. Recycled used vegetable oils may also be used. Representative examples of vegetable oils include argan oil, canola oil, rapeseed oil, coconut oil, corn oil, cottonseed oil, olive oil, palm oil, peanut oil, safflower oil, sesame oil, soy-bean oil, sunflower oil, high oleoyl soy-bean oil, high oleoyl sunflower oil, linseed oil, palm kernel oil, tung oil, castor oil, high oloeyl sunflower oil, high oleoyl soybean oil, high erucic rape oils, Jatropha oil, combinations of theses, and the like. Representative examples of animal fats include lard, tallow, chicken fat, yellow grease, fish oil, combinations of these, and the like. A representative example of a synthesized oil includes tall oil, which is a byproduct of wood pulp manufacture.

Other examples of unsaturated polyol esters include diesters such as those derived from ethylene glycol or propylene glycol, esters such as those derived from pentaerythritol or dipentaerythritol, or sugar esters such as SEFOSE®. Non-limiting examples of sucrose polyesters suitable for use include SEFOSE® 1618S, SEFOSE® 1618U, SEFOSE® 1618H, Sefa Soyate IMF 40, Sefa Soyate LP426, SEFOSE® 2275, SEFOSE® C1695, SEFOSE® C18:0 95, SEFOSE® C1495, SEFOSE® 1618H B6, SEFOSE® 1618S B6, SEFOSE® 1618U B6, Sefa Cottonate, SEFOSE® C1295, Sefa C895, Sefa C1095, SEFOSE® 1618S B4.5, all available from The Procter and Gamble Co. of Cincinnati, Ohio.

Other examples of suitable natural polyol esters may include but not be limited to sorbitol esters, maltitol esters, sorbitan esters, maltodextrin derived esters, xylitol esters, and other sugar derived esters. The chain lengths of esters are not restricted to C8-C22 and can include natural esters that come from co-metathesis of fats and oils with short chain olefins both natural and synthetic, providing an unsaturated polyol ester feedstock which can have even and odd chains as well as shorter and longer chains for the self metathesis reaction. Suitable short chain olefins include ethylene and butene.

The oligomers derived from the metathesis of unsaturated polyol esters may be further modified via hydrogenation. For example, the oligomer can be about 60% hydrogenated or more; about 70% hydrogenated or more; about 80% hydrogenated or more; about 85% hydrogenated or more; about 90% hydrogenated or more; or generally 100% hydrogenated.

The triglyceride oligomer can be derived from the self-metathesis of soybean oil. The soy oligomer can include hydrogenated soy polyglycerides. The soy oligomer may also include C₁₅-C₂₃ alkanes, as a byproduct. An example of metathesis derived soy oligomers is the fully hydrogenated DOW CORNING® HY-3050 soy wax, available from Dow Corning.

The metathesized unsaturated polyol esters can also be used as a blend with one or more non-metathesized unsaturated polyol esters. The non-metathesized unsaturated polyol esters can be fully or partially hydrogenated. Such an example is DOW CORNING® HY-3051, a blend of HY-3050 oligomer and hydrogenated soybean oil (HSBO), available from Dow Corning. The non-metathesized unsaturated polyol ester may be an unsaturated ester of glycerol. Sources of unsaturated polyol esters of glycerol include synthesized oils, natural oils (e.g., vegetable oils, algae oils, bacterial derived oils, and animal fats), combinations of theses, and the like. Recycled used vegetable oils may also be used. Representative examples of vegetable oils include those listed above.

Other modifications of the polyol ester oligomers can be partial amidation of some fraction of the esters with ammonia or higher organic amines such as dodecyl amine or other fatty amines. This modification will alter the overall oligomer composition but can be useful in some applications providing increased lubricity of the product. Another modification can be via partial amidation of a poly amine providing potential for some pseudo cationic nature to the polyol ester oligomers. Examples of such modified oligomers may be found, for example, in PCT Application Publication No. WO2012/006324 entitled “Waxes Derived from Metathesized Natural Oils and Amines and Methods of Making.” The modified oligomer may comprise, for example, DOW CORNING® HY-3200 Emulsifying Soy Wax, available from Dow Corning. In one example, the personal care composition is free of amidized polyol ester oligomers.

The polyol ester oligomers may be modified further by partial hydroformylation of the unsaturated functionality to provide one or more OH groups and an increase in the oligomer hydrophilicity.

The metathesized unsaturated polyol esters and blends can be formulated as small particle emulsions. An emulsion of the triglyceride oligomer can be prepared using a combination of non-ionic, zwitterionic, cationic, and anionic surfactants. The emulsion of the triglyceride oligomer may be a combination of non-ionic and anionic surfactants. Suitable non-ionic emulsifiers include Neodol 1-5. Suitable anionic emulsifiers include alkyl and alkyl ether sulfates having the respective formulae ROSO₃Na and RO(C₂H₄O)ₓSO₃Na. The metathesized unsaturated polyol esters can be pre-melted prior to emulsification and incorporated into the personal care composition. In some small particle emulsions, the metathesized unsaturated polyol esters can have a particle size of from about 0.05 to about 35 microns, from about 0.1 to about 10 microns, or from about 0.1 to about 2 microns.

The unsaturated polyol esters and blends can be modified prior to oligomerization to incorporate near terminal branching. Exemplary polyol esters modified prior to oligomerization to incorporate terminal branching are set forth in WO2012/009525 A2, which is incorporated herein by reference.

### III. METHODS RELATING TO THE USE OF PERSONAL CLEANSING COMPOSITIONS

While only certain personal cleansing composition components are included in this section for the sake of brevity, all such disclosure above related to the composition components may be used in the methods described below as if specifically recited in this section.

Personal cleansing compositions as described herein can provide a multitude of benefits. One benefit can be the enhancement of deposition of the benefit agent alone versus the benefit agent combined with an oligomer. For example, a method of enhancing deposition of a hydrophobic benefit agent on skin can include providing a personal cleansing composition with a benefit phase having a hydrophobic benefit agent and from about 1% to about 15%, by weight of the benefit phase, of a metathesized unsaturated polyol ester oligomer. The method of enhancing deposition of a hydrophobic benefit agent on skin can further comprise instructing an individual to apply the personal cleansing composition to the skin. The instructing step can include, for example, instructions on the package, in a flier, in a pamphlet, on literature, at the shelf, in advertising, etc. As set forth above, the hydrophobic benefit agent can exhibit a Vaughan solubility parameter from about 5 to about 14 and exhibit a viscosity of about 1500 cP or less at from about 20° C. to about 25° C. Deposition of a benefit agent, such as soybean oil, can increase by about 100% or more; about 200% or more; by about 300% or more or from about 100%; from about 200%, from about 300%, or from about 400%, to about 1000%, 2000%, 4000%, 5000%, 7500%, 10000%, or any combination thereof.

Personal cleansing compositions as described herein can also provide enhanced skin hydration. One method for enhancing skin hydration can include providing a personal cleansing composition comprising a cleansing phase and a benefit phase. As described herein, a benefit phase can include a hydrophobic benefit agent and from about 1% to about 15%, from about 1% to about 11%, from about 1% to about 10%, or from about 2% to about 10%, by weight of the benefit phase, of an oligomer. One exemplary oligomer is a soy oligomer. The method of enhancing skin hydration can further include instructing an individual to apply the personal cleansing composition to the skin. The instructing step can include, for example, instructions on the package, in a flier, in a pamphlet, on literature, at the shelf, in advertising, etc.

The level of hydration of can measured, for example, by a Corneometer. For example, skin hydration can improve by about 0.1 Corneometer Units or more; by about 0.2 Corneometer Units or more; by about 0.25 Corneometer Units or more; by about 0.3 Corneometer Units or more; by about 0.5 Corneometer Units or more; by about 0.7 Corneometer Units or more; or about 1.0 Corneometer Units or more where the personal cleansing composition includes a benefit phase with an oligomer and a benefit agent at either 3 hours after treatment or 24 hours after treatment at least once a day on at least three consecutive days. Additionally, the hydration can improve from about 0.1, 0.2, 0.25, 0.3, 0.5, 0.7, 1.0, to about 0.5, 0.7, 1.0, 1.25, 1.5, 1.75, 2, 3, 4, or any combination thereof. The method of enhancing skin hydration can further include instructing an individual to apply the personal cleansing composition to the skin. The instructing step can include, for example, instructions on the package, in a flier, in a pamphlet, on literature, at the shelf, in advertising, etc.

Another way of looking at skin hydration is with the dry skin grade. In one example, dry skin grade improves by about 0.4 units or more at about 3 hours after a last treatment, by about 0.5 units or more at about 3 hours after a last treatment, by about 0.6 units or more at about 3 hours after a last treatment, wherein the personal cleansing composition comprises a benefit phase comprising an oligomer and a benefit agent and has been applied at least once per day for 3 days or more. Additionally, the dry skin grade can improve from about 0.2, 0.3, 0.4, 0.5, 0.6, or 0.7, to about 0.5, 0.6, 0.7, 0.8, 0.9, 1.0, 2.0, 5.0, or any combination thereof. The method of enhancing skin hydration can further include instructing an individual to apply the personal cleansing composition to the skin. The instructing step can include, for example, instructions on the package, in a flier, in a pamphlet, on literature, at the shelf, in advertising, etc.

A third way of looking at skin hydration is though transepidermal water loss (TEWL). The TEWL of skin improves by about 0.5 grams of water per hour per m² or more at about 3 hours after a last treatment, by about 0.6 grams of water per hour per m² or more at about 3 hours after a last treatment, by about 0.7 grams of water per hour per m² or more at about 3 hours after a last treatment, wherein the personal cleansing composition comprises a benefit phase comprising an oligomer and a benefit agent and has been applied at least once per day for 14 days or more. In other example, the composition is applied for 21 days or more. Additionally, the TEWL may improve from about 0.2, 0.3, 0.4, 0.5, 0.6, or 0.7, to about 0.5, 0.6, 0.7, 0.8, 0.9, 1.0, 2.0, 5.0, 10.0, or any combination thereof. The method of enhancing skin hydration can further include instructing an individual to apply the personal cleansing composition to the skin The instructing step can include, for example, instructions on the package, in a flier, in a pamphlet, on literature, at the shelf, in advertising, etc.

### IV. TEST METHODS

A. Dry Skin Grade Screen and Application of Materials for Corneometer and TEWL Testing

Test subjects are screened for dry skin grade of 2.5-4.0 by trained expert graders following guidelines below. Prior to the study, subjects participate in a washout period for seven days, in which the subjects only use soap that is provided to them (e.g., soap including shea butter and no beads) and abstain from washing their legs with any other products. Subjects are also instructed to abstain from applying any leave-on products to their legs during the pre-study washout period.

Visual evaluations will be done with the aid of an Illuminated Magnifying Lamp which provides 2.75× magnification and which has a shadow-free circular fluorescent light source (General Electric Cool White, 22 watt 8″ Circline). At least 36 subjects are needed to obtain sufficient replicates for each treatment. Table 1 shows a grading scale for dry skin and lists the redness and dryness characteristics associated with each grade.

TABLE 1

| Grade* | Redness | Dryness** |
|---|---|---|
| 0.0 | No redness | Perfect skin |
| 1.0 | Barely | Patches of checking and/or slight powderiness, |
| | detectable | occasional patches of small scales may be seen, |
| | redness | distribution generalized |
| 2.0 | Slight | Generalized slight powderiness, early cracking, or |
| | redness | occasional small lifting scales may be present |
| 3.0 | Moderate | Generalized moderate powderiness and/or heavy |
| | redness | cracking and lifting scales |
| 4.0 | Heavy or | Generalized heavy powderiness and/or heavy |
| | substantial | cracking and lifting scales |
| | redness | |
| 5.0 | Severe | Generalized high cracking and lifting scales, |
| | redness | eczematous change may be present, but not |
| | | prominent, may see bleeding cracks |
| 6.0 | Extreme | Generalized severe cracking, bleeding cracks and |
| | redness | eczematous changes may be present, large scales |
| | | may be sloughing off |

*Half-unit grades may be used if necessary
**“Generalized” refers to situations where more than 50% of an application area is affected

Before initial visual grading, a clinical assistant will mark 2-7 cm (across)×10 cm (down) treatment sites on an outer portion of the lower legs using a template and a laboratory marking pen (4 corner brackets are sufficient to delineate each area). For assignment of the products, two sites located on the left leg will be numbered L1 and L2, where L1 is the top part of the lower leg nearest the knee, and L2 is the bottom part of the lower leg nearest the ankle. Two sites located on the right leg will be numbered R1 and R2, where R1 is the top part of the lower leg nearest the knee, and R2 is the bottom part of the lower leg nearest the ankle.

To simplify the treatment process, master trays will be prepared for each treatment plan specified in the study randomization. Each master tray will be divided in half, with each half labeled ‘left’ or ‘right’ to indicate which leg it corresponds to, then subdivided into sections for the test products in the order of leg application site. One or more make-up trays can also be prepared for use as needed using individual coded containers, or other appropriate product code indicators, that can be re-arranged according to a given treatment plan.

Trained clinical assistants will wash each subject's lower legs in a controlled manner with assigned treatments once daily for 21 consecutive days. Assignment of test treatments to skin sites on the left and right legs will be designated by study randomization. A target dose of body wash for each site is 10 μL/cm². All body wash products will be dispensed at 0.7 mL dosages. All body wash test products will be drawn up into syringes at the 0.7 mL dosage. A one day supply of syringes for all products may be filled the day before or the day of use. Product that has been transferred to another container and the container itself will be used for one day only (i.e., the day the transfer occurred). All syringe filling operations will be appropriately documented (e.g., product code filled, when filled, initials of person responsible for filling).

The treatment area on the top part of the left leg of the subject is wetted for 5 seconds with 95-100° F. running tap water. The water flow rate is about 1200 mL per minute. For the “No Treatment” site, apply water only. For a treatment site, dispense 0.7 mL of body wash product from the syringe onto the center of the treatment area and place a wet puff over the dispensed product and gently rub the puff back and forth within the treatment site for 10 seconds. Then, allow lather (or water only) to remain on the site for 90 seconds. When residence time for a site has expired, the site is rinsed for 15 seconds under a running tap, taking care not to rinse adjacent sites. After the application area has been rinsed, the area is gently patted dry. Repeat the procedure for the lower part of the left leg, and after completion, use the same procedure for each of the top part of the right leg and the lower part of the right leg.

B. Corneometer Testing

Once the materials are applied as noted above in Section A, improvements in skin hydration can be measured with a Corneometer, while baseline measurements are taken prior to application of materials. In particular, skin hydration based upon measurements of capacitance can be assessed using the Corneometer® 825. Such use of a Corneometer is further described in U.S. patent application Ser. No. 13/007,630. Such measurements can be non-invasive and can be taken in duplicate on each site of the subjects' legs at the following times: At baseline, prior to 1^{st} treatment; 3 hours post 1^{st}, 3^{rd}, 5^{th} 14^{th} and 21^{st} treatments; 24 hours post 4^{th}, 13^{th} and 21^{st}, treatments, 48 hours post 21^{st} treatment after a visual assessment has been completed. Subjects can be acclimated for a minimum of thirty minutes in an environmentally controlled room (maintained at 70° F.±2 and 30-45% relative humidity) prior to the non-invasive instrumental measurements taken on their legs. Data can be recorded electronically using a Sponsor's direct data entry and data capture programs. Measurements can be performed according to a test facility's standard operating procedures and/or the Sponsors Instrument Operation Manual.

The Corneometer values are arbitrary units for electrical impedance. At baseline, for subjects having a dry skin grade from about 2.5 to about 4.0, an adjusted mean of such Corneometer values can typically fall within a range of about 15 to about 20. Higher Corneometer values can correspond to a higher hydration level, and thus, lower Corneometer values can correspond to lower hydration levels.

The instrument should only be operated by trained operators. Further, the same instrument(s) and operator(s) can be used throughout the study. Kimwipes can be used to wipe an end of a probe. The probe can be wiped with a Kimwipe between each measurement. At the end of an evaluation session, data collected for that period can be backed up according to instructions in the Sponsors Instrument Operation Manual, and a hard copy of the data can be printed.

C. Transepidermal Water Loss (TEWL) Method

Once the materials are applied as noted above in Section A, the step of assessing erythema and/or dryness by objective instrumental measurements may include evaluating the portion of skin with a transepidermal water loss instrument, commercially available from Cortex Technology, Denmark under the tradename TEWL, DermaLab® Evaporimeter. Participants may be conditioned in a temperature and humidity controlled room (73° F.±4° F. (about 23° C.±2.2° C.) and a relative humidity of 50%±10%) for approximately 20 minutes.

D. In-Vitro Deposition Evaluation Method

The in-vitro Deposition Evaluation Method measures the deposition of benefit agents on a skin mimic The method compares the quantity of benefit agent of the skin mimic surface before and after cleansing in an automated cleansing unit, such as the automated cleansing unit described in co-pending and co-assigned Multiphase Personal Care Composition With Enhanced Deposition, U.S. application Ser. No. 12/510,880 (filed Jul. 28, 2009) and In-Vitro Deposition Evaluation Method for Identifying Personal Care Compositions Which Provide Improved Deposition of Benefit Agents, U.S. application Ser. No. 12/511,034 (filed Jul. 28, 2009).

The in-vitro Deposition Evaluation Method uses two 12-well plates (hereinafter referred to as “plates”). Suitable 12-well plates are commercially available from Greiner bio-one. For example, the Cellstar® 12-well suspension culture plate has 3 rows and 4 columns with a well volume of about 6.2 mL. The Cellstar® 12-well suspension culture plate comprises the approximate dimensions of 19 mm in height, 127 mm in length and 85 mm in width. The Cellstar® 12-well suspension culture plate has a well diameter of 23 mm, a well depth of 15 and a well to well spacing of 2 mm A Cellstar® 12-well suspension culture plate is provided for containing the samples comprising the personal cleansing composition as described in the Examples herein.

The in-vitro Deposition Evaluation Method uses approximately 120 g of bodies for two plates. Five grams of bodies carefully loaded into each of the 12 wells of the two plates to ensure the same quantity is loaded into each well. Each body is a spherical stainless steel bearing that is approximately 2 mm in circumference. Each body comprises ferrometallic material. Suitable bodies are those available from WLB Antriebeselemente Gmbh, Scarrastrasse 12, D-68307 Mannheim, Germany.

The personal cleansing compositions can be prepared as described by the examples herein. After the examples of the personal cleansing compositions are prepared, control and test samples are prepared by determining the dilution ratio and dispensing both the personal cleansing composition and distilled water into the wells of the microplate and allow the samples to mix while being exposed to the automated washing process. The dilution ratio used in this application is one part of composition and twenty nine parts of water (1:29). A pre-calibrated positive displacement pipette is used to dispense 66.7 μL of composition on to the bodies in each well, followed by dispensing 1933.3 μL of distilled water into each well. The control samples and test samples are dispensed in the specified wells of the plate, all within a 20-minute time frame. Each composition is placed in 6 different well, 3 of which are in plate 1 and the other 3 well are in plate 2. A test control composition containing the benefit agent should be used in every test to ensure consistency among tests.

The skin mimic used in the in-vitro Deposition Evaluation Method is comprised of a molded bicomponent polyurethane substrate. The skin mimic is textured on one side with a pattern that resembles the texture of human skin. The textured side of the skin mimic is coated with 1,1,1-trimethyl-1-pentene that is plasma deposited. The skin mimic surface has a total surface energy of 32±1.0 (mJ/m²) and a contact angle in water of 100°±2.0. Suitable skin mimic surface materials are described in co-pending and co-assigned Coated Substrate with Properties of Keratinous Tissue, U.S Patent Pub. No. 20070128255A1 (filed Aug. 11, 2006) (published Jun. 7, 2007) and Methods of Use of Substrate Having Properties of Keratinous Tissue, U.S Patent Pub. No. 20070288186A1 (filed Feb. 5, 2007) (published Dec. 13, 2007).

After all of the wells of the plate are filled with the samples and the pieces of skin are made and coated, the skin mimic is prepared for the in-vitro Deposition Evaluation Method. Two pieces of skin mimic are prepared by cutting the skin mimic to fit on top of all 12 openings of the wells of the plate while wearing gloves. The two pieces of skin mimic pieces are numbered “1” and “2.”

Next, the pieces of skin mimics are arranged over the openings of the wells of the microplates. The pieces of skin mimic surface material are transferred to cover the openings of the wells of the each of the plates to ensure that the textured and treated region of the skin mimic is facing the openings of the wells of the plate. A lid is placed over each piece of the skin mimic and the associated plate to form a lidded plate.

The lidded plates are placed into plate holders of an automated cleansing unit, or, a device used in the in-vitro Deposition Evaluation Method of the present invention. The automated cleansing unit comprises a horizontal base comprising four microplate holders. The horizontal base is made of rectangle of aluminum comprising the following approximate dimensions of ⅜ inch in height, fourteen inches in width and twenty seven inches in length. The automated cleansing unit further comprises two vertical supports comprised of aluminum with the approximate dimensions of one inch by two inches by ten and ¾ of an inch in height. The vertical supports are attached to a horizontal support comprising a rodless air slide. The horizontal support comprising a rodless air slide comprises the approximately dimension of a ½ inch by two inches by twenty six and ½ inches in height. Suitable rodless air slides comprise a one inch bore and eleven inch stroke and have associated end lugs and mount brackets, which are commercially available from McMaster-Carr. The rodless air slide can be double acting and comprises a carriage that is connected to an internal piston and two compressed air ports.

The automated cleansing unit comprises two magnetic arms. The horizontal support comprising a rodless air slide is the structure upon which the two magnetic arms are mounted. The magnetic arms are mounted to the rodless air slide such that the magnetic arms move back and forth along the length of the double acting rodless air slide by the force of compressed air. Each of the magnetic arms are comprised of aluminum and have the approximate dimensions of one inch by two inches by fourteen inches in length and have a “T” shape channel that houses seven neodymium iron boron magnets (not shown). Each of the neodymium iron boron magnets has the approximate dimensions of two inches in length, one inch in width and half or an inch in height. Each of the neodymium iron boron magnets comprises a magnetic strength of 12200 Gauss, available from Edmund Scientifics. The magnetic arms are configured at a height of about 2.75 cm above the microplate holder with the caveat that the magnets maintain their function to attract and move the bodies comprised within the wells of the microplate. The magnetic arms move back and forth along the length of the rodless air slide by the force of compressed air at a speed of approximately 6 back and forth sweeps over the length of the rodless air slide over a 10 second time period.

The magnetic arms can be configured with four microplate holders. Each of the microplate holders comprise a clamping plate and four pistons attached to a pneumatic control unit. When actuated, the pistons for the pneumatic control unit hold the plates in the four plate holders at a pressure of about 90 psi. Prior to placing the lidded plates into the plate holders of automated cleansing unit, the pneumatic control unit is turned on.

The automated cleansing unit can comprise a pneumatic control unit. The top view shows components of the pneumatic control unit which can be connected to the rodless air slide, the piston and clamping plates. The pneumatic control unit can be used to apply compressed air to the automated cleansing unit, which imparts a force by converting the potential energy of compressed air into kinetic energy. The pneumatic control unit comprises a solenoid air control valve, a distribution manifold outlet, a compressed air control valve, a compressed air flow regulator, an alternating output binary valve, a two-hand safety pneumatic control valve, a compressed air control valve and various connectors that provide pressurized air to the automated cleansing unit from an external air source. The air control valve, air flow regulators, alternating a binary valves, a two-hand safety pneumatic control valve are positioned upstream of a solenoid air control valve. An exemplary suitable solenoid air control valve as used herein has a double air style valve with a 10 psi to 120 psi operating pressure. Suitable compressed air flow regulators can operate in the pressure range of 14 psi to 116 psi. Suitable air control valve alternating output binary valves can operate in a 35 psi to 100 psi range. All of the components of the pneumatic control unit are available from McMaster-Carr®.

The lidded plates are placed into the plate holders and pneumatic control unit is actuated such that the lidded plates are held under 90 psi of pressure. The magnetic arms are actuated on and arms moves over the lidded microplates at a height of 2.65 cm above the plate holders. The magnetic arms of the automated cleansing unit, sweep back and forth over the plate holders for 5 minutes, at a speed of 6 sweeps per every 10 seconds. After 5 minutes of the automated cleansing process, the lidded plates are removed from the plate holders and are disassembled.

After the automated washing process, two large 4000 mL beakers of 20° C. to 25° C. water are filled. The first piece of skin mimic is removed from the first plate and submerged in the tap water within the first beaker five times. The second piece of skin mimic is removed from the second microplate and submerged within the second beaker five times. The completeness of rinsing step is judged visually by the lack of foam on the skin mimic and presence of defined circles of deposited material on the skin mimic Both piece of skin mimic are blotted gently with paper towels and fumed in a drying hood for at least 3 hours each.

Clean the blades of the 12-well die with alcohol and Q-tips and the clear cutter plate with Dawn & tap water. Dry the clear cutter plate with paper towels. Next, gently place the first piece of skin mimic, deposit side down, onto the 12-well die, lining up the deposit sites with the circle blades. Gently place the clear cutter lid over the first piece of skin mimic, again lining up the circles of the lid with the deposit sites & circle blades below, and then place this whole die unit into the pneumatic cutter. Operate the cutter dual-switch with both hands, holding for a few seconds to ensure a good cut. Remove the die unit, and then gently lift the clear lid up and over to examine the cut mimic pieces.

Position the labeled glass vials nearby to correspond with the position of the mimic on the clear lid, according to rows & columns labeled previously on the mimic Using a clean straight pin, poke each deposit circle site and transfer to the appropriate vial, capping each vial upon transferring. Follow the same procedure for the second piece of skin mimic The cut-out pieces of treated skin mimic are then extracted with a solvent and the extract is analyzed and quantified. Add 50 μL of internal standard and 5 mL of 50:50 isopropyl alcohol:heptane to the cut-out pieces of skin mimic in the 20 mL glass vial. Cap the vial tightly and vortex at 1500 rpm (pulse) for 10 minutes. Transfer extract to autosampler vial. Gas chromatography analysis was conducted using an Agilent 6890, or an equivalent device with a split/splitless capillary inlet system, flame ionization detector, and data system. A gas chromatography column of Agilent DB-1HT, 15 M×0.25 mm ID, 0.10 μm film thickness or equivalent was used.

E. Benefit Phase Rheology Method

The rheological properties of the benefit phase are measured on a stress controlled rheometer, such as the AR1000 stress rheometer by TA Instrument, using 40 millimeter stainless steel parallel plates with 1 millimeter gap. Smaller plates, such as 25 mm, may be used if the benefit phase is significantly rigid. 1 mL of a sample is placed onto the lower plate. The upper plate is lowered at a Normal Force setting (maximum Normal Force is 50 N) and a compression velocity of 100 μm/s. The excess material is trimmed using a plastic flat edge ensuring that material is not sheared by movements of the plates. A stress sweep is run logarithmically between 0.1-1000 Pa at an angular frequency of 1 radians/second. Data is collected at 10 points/decade in log mode. The storage shear modulus (G′) and the loss shear modulus (G″) are plotted as a function of oscillatory stress on a log-log scale. The oscillatory stress at which G′ and G″ are equal is recorded as the crossover stress. For most solid-like materials, the G′ and G″ curves will form a plateau at low stresses, forming a region known as a linear viscoelastic region (LVR), which defines a stress window in which a material's structure remains intact. When the solid-like material is subjected to a stress above the LVR, the material's structure is irreversibly changed and gross deformation occurs.

### V. EXAMPLES

### A. Comparative Examples 1-7

For Comparative Examples 1-7, personal cleansing compositions are formed with a benefit phase including RBD (refined, bleached and deodorized) soybean oil. Compositional information with respect to Comparative Examples 1-7 can be found in Table 2. The cleansing phase for each of Comparative Examples 1-7 was prepared by adding water in a mixing vessel. Then the following ingredients were added with continuously mixing: sodium chloride, water soluble cationic polymer (guar hydroxypropyltrimonium chloride, N-Hance 3196 CG-17), laurylamidopropyl betaine, sodium trideceth sulfate, sodium tridecyl sulfate, ethoxylated tridecyl alcohol, Dissolvine na3 s, sodium benzoate, and AQUPEC® SER W-300C. Adjust the pH by adding hydrogen peroxide (50% solution) to pH=5.7±0.2. Add methyl chloro isothiazolinone and methyl isothiazolinone, and mix until homogeneous. The benefit phase for each of Comparative Examples 1-7 was prepared by heating the lipids until they melt and mixing until homogeneous. Soybean oil was added into the surfactant phase through a SpeedMixer™ at a speed of 1,000 rpm for 60 seconds. For any examples where the benefit phase contains soybean oil and a wax, the benefit phase was warmed and added to a warmed surfactant phase (˜70° C.). The mixture was then mixed on a stand mixer until the composition is cooled to room temperature.

TABLE 2

| | | | | | | | |
|---|---|---|---|---|---|---|---|
| | Comparative | Compar. | Compar. | Compar. | Compar. | Compar. | Compar. |
| Ingredient/Property | Example 1 | Example 2 | Example 3 | Example 4 | Example 5 | Example 6 | Example 7 |

Cleansing Phase (amts. by wt. % of cleansing phase)

| | | | | | | | |
|---|---|---|---|---|---|---|---|
| Distilled Water | Q.S. | Q.S. | Q.S. | Q.S. | Q.S. | Q.S. | Q.S. |
| Sodium Tridecyl Ether | 12.6 | 12.6 | 12.6 | 12.6 | 12.6 | 12.6 | 12.6 |
| Sulfate (64% active) | | | | | | | |
| Laurylamidopropyl | 7.67 | 7.67 | 7.67 | 7.67 | 7.67 | 7.67 | 7.67 |
| Betaine (36.8% active) | | | | | | | |
| Sodium Chloride | 4.75 | 4.75 | 4.75 | 4.75 | 4.75 | 4.75 | 4.75 |
| Iconol TDA3- | 1.4 | 1.4 | 1.4 | 1.4 | 1.4 | 1.4 | 1.4 |
| Ethoxylated Tridecyl | | | | | | | |
| Alcohol | | | | | | | |
| Water-soluble cationic | 0.42 | 0.42 | 0.42 | 0.42 | 0.42 | 0.42 | 0.42 |
| polymer | | | | | | | |
| Sodium Benzoate, NF | 0.28 | 0.28 | 0.28 | 0.28 | 0.28 | 0.28 | 0.28 |
| Methylchloroisothiazolinone/ | 0.037 | 0.037 | 0.037 | 0.037 | 0.037 | 0.037 | 0.037 |
| methylisothiaxolinone | | | | | | | |
| AQUPEC ® SER W-300C | 0.15 | 0.15 | 0.15 | 0.15 | 0.15 | 0.15 | 0.15 |
| Dissovine na2-s | 0.15 | 0.15 | 0.15 | 0.15 | 0.15 | 0.15 | 0.15 |
| Hydrogen peroxide | 0.07 | 0.07 | 0.07 | 0.07 | 0.07 | 0.07 | 0.07 |
| solution, 50% | | | | | | | |

Benefit Phase (amts. by wt. % of benefit phase)

| | | | | | | | |
|---|---|---|---|---|---|---|---|
| RBD Soybean Oil | 100 | 90 | 90 | 90 | 100 | 100 | 100 |
| Soy wax | — | 10 | — | — | — | — | — |
| Beeswax | — | — | 10 | — | — | — | — |
| Paraffin | — | — | — | 10 | — | — | — |
| Cleansing phase:Benefit | 90:10 | 90:10 | 90:10 | 90:10 | 95:5 | 98:2 | 85:15 |
| phase Ratio | | | | | | | |
| in vitro Soybean oil | 13 | 22 | 24 | 4 | 6 | 5 | 16 |
| deposition (μg/cm²) | | | | | | | |

### B. Inventive Examples 8-13

For Inventive Examples 8-13, personal cleansing compositions are formed with a benefit phase including RBD soybean oil and a soy oligomer. Compositional information with respect to Inventive Examples 8-13 can be found in Table 3. The cleansing phase for each of Inventive Examples 8-13 was prepared by adding water in a mixing vessel. Then the following ingredients were added with continuously mixing: sodium chloride, water soluble cationic polymer (guar hydroxypropyltrimonium chloride, N-Hance 3196 CG-17), laurylamidopropyl betaine, sodium trideceth sulfate, sodium tridecyl sulfate, ethoxylated tridecyl alcohol, Dissolvine na3 s, sodium benzoate, and AQUPEC® SER W-300C. Adjust the pH by adding hydrogen peroxide (50% solution) to pH=5.7±0.2. Add methyl chloro isothiazolinone and methyl isothiazolinone, and mix until homogeneous. The benefit phase for each of Inventive Examples 8-13 was prepared by heating the lipids until they melt and mixing until homogeneous. Soybean oil was added into the surfactant phase through a SpeedMixer™ at a speed of 1,000 rpm for 60 seconds. For any examples where the benefit phase contains soybean oil and a soy oligomer, the benefit phase was warmed to allow sufficient mixing and added to a warmed surfactant phase (˜70° C.). The mixture was then mixed through a SpeedMixer™ or on a stand mixer until the composition is cooled to room temperature.

TABLE 3

| | | | | | | |
|---|---|---|---|---|---|---|
| | Inventive | Inventive | Inventive | Inventive | Inventive | Inventive |
| Ingredient/Property | Example 8 | Example 9 | Example 10 | Example 11 | Example 12 | Example 13 |

Cleansing Phase (amts. by wt. % of cleansing phase)

| | | | | | | |
|---|---|---|---|---|---|---|
| Distilled Water | Q.S. | Q.S. | Q.S. | Q.S. | Q.S. | Q.S. |
| Sodium Tridecyl Ether | 12.6 | 12.6 | 12.6 | 12.6 | 12.6 | 12.6 |
| Sulfate (64% active) | | | | | | |
| Laurylamidopropyl | 7.67 | 7.67 | 7.67 | 7.67 | 7.67 | 7.67 |
| Betaine (36.8% active) | | | | | | |
| Sodium Chloride | 4.75 | 4.75 | 4.75 | 4.75 | 4.75 | 4.75 |
| Iconol TDA3- | 1.4 | 1.4 | 1.4 | 1.4 | 1.4 | 1.4 |
| Ethoxylated Tridecyl | | | | | | |
| Alcohol | | | | | | |
| Water-soluble cationic | 0.42 | 0.42 | 0.42 | 0.42 | 0.42 | 0.42 |
| polymer | | | | | | |
| Sodium Benzoate, NF | 0.28 | 0.28 | 0.28 | 0.28 | 0.28 | 0.28 |
| Methylchlorois othiazolinone/ | 0.037 | 0.037 | 0.037 | 0.037 | 0.037 | 0.037 |
| methylisothiaxolinone | | | | | | |
| AQUPEC ® SER W-300C | 0.15 | 0.15 | 0.15 | 0.15 | 0.15 | 0.15 |
| Dissovine na2-s | 0.15 | 0.15 | 0.15 | 0.15 | 0.15 | 0.15 |
| Hydrogen peroxide | 0.07 | 0.07 | 0.07 | 0.07 | 0.07 | 0.07 |
| solution, 50% | | | | | | |

Benefit Phase (amts. by wt. % of benefit phase)

| | | | | | | |
|---|---|---|---|---|---|---|
| RBD Soybean Oil | 90 | 95 | 97.5 | — | 90 | 90 |
| Soy oligomer (DCHY3050) | 10 | 5 | 2.5 | — | 10 | 10 |
| Hydrogenated soybean | — | — | — | 100 | — | — |
| oil and soy oligomer | | | | | | |
| (10%) blend (DCHY3051) | | | | | | |
| Cleansing phase:Benefit | 90:10 | 90:10 | 90:10 | 85:15 | 95:5 | 98:2 |
| phase Ratio | | | | | | |
| in vitro Soybean oil | 731 | 552 | 72 | 1037 | 234 | 47 |
| deposition (μg/cm²) | | | | | | |

### C. Comparative Example 14 and Inventive Example 15

For Comparative Example 14 and Inventive Example 15, personal cleansing compositions are formed with a benefit phase including a sucrose polyester. Inventive Example 15 further includes a soy oligomer in the benefit phase. Compositional information with respect to Comparative Example 14 and Inventive Example 15 can be found in Table 4. The cleansing phase for each of Comparative Example 14 and Inventive Example 15 was prepared by adding water in a mixing vessel. Then the following ingredients were added with continuously mixing: sodium chloride, water soluble cationic polymer (guar hydroxypropyltrimonium chloride, N-Hance 3196 CG-17), laurylamidopropyl betaine, sodium trideceth sulfate, sodium tridecyl sulfate, ethoxylated tridecyl alcohol, Dissolvine na3 s, sodium benzoate, and AQUPEC® SER W-300C. Adjust the pH by adding hydrogen peroxide (50% solution) to pH=5.7±0.2. Add methyl chloro isothiazolinone and methyl isothiazolinone, and mix until homogeneous. The benefit phase for each of Comparative Example 14 and Inventive Example 15 was prepared by heating the lipids until they melt and mixing until homogeneous. Soybean oil was added into the surfactant phase through a SpeedMixer™ at a speed of 1,000 rpm for 60 seconds.

TABLE 4

| | | |
|---|---|---|
| | Comparative | Inventive |
| Ingredient/Property | Example 14 | Example 15 |

Cleansing Phase (amts. by wt. % of cleansing phase)

| | | |
|---|---|---|
| Distilled Water | Q.S. | Q.S. |
| Sodium Tridecyl Ether Sulfate (64% | 12.6 | 12.6 |
| active) | | |
| Laurylamidopropyl Betaine (36.8% active) | 7.67 | 7.67 |
| Sodium Chloride | 4.75 | 4.75 |
| Iconol TDA3-Ethoxylated Tridecyl | 1.4 | 1.4 |
| Alcohol | | |
| Water-soluble cationic polymer | 0.42 | 0.42 |
| Sodium Benzoate, NF | 0.28 | 0.28 |
| Methylchloroisothiazolinone/methyliso- | 0.037 | 0.037 |
| thiaxolinone | | |
| AQUPEC ® SER W-300C | 0.15 | 0.15 |
| Dissovine na2-s | 0.15 | 0.15 |
| Hydrogen peroxide solution, 50% | 0.07 | 0.07 |

Benefit Phase (amts. by wt. % of benefit phase)

| | | |
|---|---|---|
| Sucrose polyester (1618S) | 100 | 90 |
| Soy oligomer (DC HY3050) | — | 10 |
| Surfactant phase:lipid phase ratio in | 90:10 | 90:10 |
| compositions | | |
| in vitro Sucrose polyester deposition | 53 | 264 |
| (μg/cm²) | | |

Table 5 shows results for a transepidermal water loss (TEWL) test for a personal cleansing composition having a cleansing phase-benefit phase ratio of 85:15, where the benefit phase includes soybean oil (Comparative Example 7) and a personal cleansing composition having a cleansing phase-benefit phase ratio of 85:15, where the benefit phase includes soybean oil and about 10%, by weight of the benefit phase, of a soy oligomer (Inventive Example 11). Results from this test are based on measurements taken 3 hours after the last treatment. The TEWL test is described above. As illustrated, after 14 days, treatment with Inventive Example 11, with the benefit phase having a soy oligomer, exhibits a lower water loss relative to Comparative Example 7.

TABLE 5
TEWL Test Results, 3 Hours After Last Treatment

| | | Inventive Example 11 |
|---|---|---|
| Days (measured at | Comparative Example 7 | (with Soybean Oil and |
| 3 hours after | (with Soybean Oil), g of | Soy Oligomer), g of |
| last treatment) | water per hour per m² | water per hour per m² |
| 0 | 4.622 | 4.613 |
| 3 | 4.624 | 4.552 |
| 5 | 4.952 | 5.268 |
| 14 | 6.141 | 5.356 |
| 21 | 6.482 | 5.606 |

Table 6 shows results for a transepidermal water loss (TEWL) test for a personal cleansing composition having a cleansing phase-benefit phase ratio of 85:15, where the benefit phase includes soybean oil (Comparative Example 7) and a personal cleansing composition having a cleansing phase-benefit phase ratio of 85:15, where the benefit phase includes soybean oil and about 10%, by weight of the benefit phase, of a soy oligomer (Inventive Example 11). Results from this test are based on measurements taken 24 hours after the last treatment (the measurement for Day 23 is 48 hours after the 21^{st} treatment). The TEWL test is described above. As illustrated, after 14 days, treatment with Inventive Example 11, with the benefit phase having a soy oligomer, exhibits a lower water loss relative to Comparative Example 7.

TABLE 6
TEWL Test Results, 24 Hours After Last Treatment

| | | Inventive Example 11 |
|---|---|---|
| Days (measured at | Comparative Example 7 | (with Soybean Oil and |
| 24 hours after | (with Soybean Oil), g of | Soy Oligomer), g of |
| last treatment) | water per hour per m² | water per hour per m² |
| 0 | 4.622 | 4.613 |
| 5 | 5.090 | 5.260 |
| 14 | 6.281 | 5.637 |
| 22 | 6.381 | 5.809 |
| 23* | 6.109 | 5.695 |

*Day 23 is 48 hours after 21^{st} treatment

Table 7 shows results for a Corneometer test for a personal cleansing composition having a cleansing phase-benefit phase ratio of 85:15, where the benefit phase includes soybean oil (Comparative Example 7) and a personal cleansing composition having a cleansing phase-benefit phase ratio of 85:15, where the benefit phase includes soybean oil and about 10%, by weight of the benefit phase, of a soy oligomer (Inventive Example 11). Results from this test are based on measurements taken 3 hours after the last treatment. The Corneometer test is described above. As illustrated, treatment with Inventive Example 11, with the benefit phase having a soy oligomer, exhibits higher Corneometer values relative to Comparative Example 7, and thus a greater hydration level.

TABLE 7
Corneometer Test Results, 3 Hours After Last Treatment

| Days (measured at | | Inventive Example 11 |
|---|---|---|
| 3 hours after | Comparative Example 7 | (with Soybean Oil and |
| last treatment) | (with Soybean Oil) | Soy Oligomer) |
| 0 | 18.472 | 18.814 |
| 1 | 19.116 | 19.733 |
| 3 | 20.096 | 20.673 |
| 5 | 19.121 | 20.034 |
| 14 | 18.238 | 18.665 |
| 21 | 15.633 | 16.922 |

Table 8 shows results for a Corneometer test for a personal cleansing composition having a cleansing phase-benefit phase ratio of 85:15, where the benefit phase includes soybean oil (Comparative Example 7) and a personal cleansing composition having a cleansing phase-benefit phase ratio of 85:15, where the benefit phase includes soybean oil and about 10%, by weight of the benefit phase, of a soy oligomer (Inventive Example 11). Results from this test are based on measurements taken 24 hours after the last treatment (the measurement for Day 23 is 48 hours after the 21^{st} treatment). The Corneometer test is described above. As illustrated, treatment with Inventive Example 11, with the benefit phase having a soy oligomer, exhibits higher Corneometer values relative to Comparative Example 7, and thus a greater hydration level.

TABLE 8
Corneometer Test Results, 24 Hours After Last Treatment

| Days (measured at | | Inventive Example 11 |
|---|---|---|
| 24 hours after | Comparative Example 7 | (with Soybean Oil and |
| last treatment) | (with Soybean Oil) | Soy Oligomer) |
| 0 | 18.472 | 18.814 |
| 5 | 20.122 | 20.542 |
| 14 | 17.417 | 18.470 |
| 22 | 15.582 | 16.090 |
| 23* | 15.631 | 16.826 |

*Day 23 is 48 hours after 21^{st} treatment

Table 9 shows results for a dry skin grade test for a personal cleansing composition having a cleansing phase-benefit phase ratio of 85:15, where the benefit phase includes soybean oil (Comparative Example 7) and a personal cleansing composition having a cleansing phase-benefit phase ratio of 85:15, where the benefit phase includes soybean oil and about 10%, by weight of the benefit phase, of a soy oligomer (Inventive Example 11). Results from this test are based on measurements taken 3 hours after the last treatment. The dry skin grade test is described above. As illustrated, after 3 days, treatment with Inventive Example 11, with the benefit phase having a soy oligomer, exhibits a lower dry skin grade level relative to Comparative Example 7, and thus a greater hydration level.

TABLE 9
Dry Skin Grade Test Results, 3 Hours After Last Treatment

| Days (measured at | | Inventive Example 11 |
|---|---|---|
| 3 hours after | Comparative Example 7 | (with Soybean Oil and |
| last treatment) | (with Soybean Oil) | Soy Oligomer) |
| 0 | 3.094 | 2.981 |
| 1 | 2.130 | 2.246 |
| 3 | 2.585 | 1.961 |
| 5 | 2.598 | 1.774 |
| 14 | 2.882 | 1.540 |
| 21 | 3.468 | 2.217 |

Table 10 shows results for a dry skin grade test for a personal cleansing composition having a cleansing phase-benefit phase ratio of 85:15, where the benefit phase includes soybean oil (Comparative Example 7) and a personal cleansing composition having a cleansing phase-benefit phase ratio of 85:15, where the benefit phase includes soybean oil and about 10%, by weight of the benefit phase, of a soy oligomer (Inventive Example 11). Results from this test are based on measurements taken 24 hours after the last treatment (the measurement for Day 23 is 48 hours after the 21^{st} treatment). The dry skin grade test is described above. As illustrated, treatment with Inventive Example 11, with the benefit phase having a soy oligomer, exhibits a lower dry skin grade level relative to Comparative Example 7, and thus a greater hydration level.

TABLE 10
Dry Skin Grade Test Results, 24 Hours After Last Treatment

| Days (measured at | | Inventive Example 11 |
|---|---|---|
| 24 hours after last | Comparative Example 7 | (with Soybean Oil and |
| treatment) | (with Soybean Oil) | Soy Oligomer) |
| 0 | 3.094 | 2.981 |
| 5 | 3.151 | 2.863 |
| 14 | 3.337 | 2.858 |
| 22 | 3.847 | 3.468 |
| 23* | 3.702 | 3.464 |

*Day 23 is 48 hours after 21^{st} treatment

### D. Inventive Non-Aerosol Shave Preparation Examples 16-22

Non-aerosol shave preparation examples 16-22 are prepared by weighing out the water and glycerin in a vessel sufficient to hold the entire batch. Insert an overhead mixer with impeller into the vessel and increase agitation to create a vortex. Pre-blend the cellulose thickener and PEG polymer powders then sprinkle the polymer blend into the vortex until dispersed. Reduce agitation to avoid aeration. Begin heating the batch to 80° C. Add the fatty acids, glyceryl oleate, and oligomer blend. Mix until uniform and melted. Continue heating to 80° C. Once the batch is at least 80° C., add the TEA and mix until uniform and dispersed. Add the potassium hydroxide and mix until uniform and dispersed. Begin cooling batch to below 45° C. Once below 45° C., add the perfume, preservatives, soap, and other temperature-sensitive additives. Cool to below 35° C. and QS with water.

| | Example | Example | Example | Example | Example | Example | Example |
|---|---|---|---|---|---|---|---|
| | 16 | 17 | 18 | 19 | 20 | 21 | 22 |
| Water | Q.S. | Q.S. | Q.S. | Q.S. | Q.S. | Q.S. | Q.S. |
| Glycerine | 12.50% | 0.50% | 2.50% | 2.50% | 2.50% | 10.00% | 2.50% |
| PEG 90M | 0.06% | 0.10% | 0.10% | 0.04% | 0.04% | 0.06% | 0.05% |
| PEG 23M | 0.04% | 0.00% | 0.00% | 0.05% | 0.05% | 0.05% | 0.04% |
| Hydroxyethylcellulose | 0.50% | 0.50% | 0.50% | 0.50% | 0.50% | 0.41% | 0.41% |
| Palmitic acid | 10.50% | 14.00% | 10.50% | 14.00% | 8.40% | 14.00% | 10.50% |
| Stearic Acid | 7.50% | 10.00% | 7.50% | 10.00% | 6.00% | 10.00% | 7.50% |
| Glyceryl Oleate | 1.25% | 1.66% | 1.25% | 0.00% | 1.00% | 1.50% | 1.25% |
| HY-3051 Soy Wax | 5.00% | 5.00% | 5.00% | 7.50% | 7.50% | 10.00% | 10.00% |
| Blend | | | | | | | |
| Sorbitol | 1.00% | 2.00% | 1.00% | 2.00% | 1.00% | 1.00% | 1.00% |
| Triethanolamine | 6.75% | 9.00% | 6.75% | 9.00% | 5.40% | 9.00% | 6.75% |
| Potassium Hydroxide | 1.30% | 1.80% | 1.30% | 1.80% | 1.00% | 1.80% | 1.31% |
| Other (perfume, etc.) | 1.00% | 0.60% | 1.00% | 1.00% | 1.30% | 1.30% | 1.00% |
| DMDM Hydantoin | | | | | | | |
| and Iodopropynyl | | | | | | | |
| Butylcarbamate | 0.25% | 0.25% | 0.25% | 0.25% | 0.25% | 0.25% | 0.25% |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of enhancing deposition of a hydrophobic benefit agent on skin, comprising:
providing a rinse-off personal cleansing composition comprising a cleansing phase comprising a surfactant and water and a benefit phase comprising a benefit agent and from about 1% to about 15% of an oligomer derived from metathesis of unsaturated polyol esters, and
instructing an individual to apply the composition to the skin.

2. The method of claim 1, wherein the deposition improves 100% or more versus the personal care composition without the oligomer where deposition is measured in accordance with the In-vitro Deposition Evaluation model.

3. The method of claim 1, wherein the deposition improves from about 100% to about 10,000% versus the benefit agent alone where deposition is measured in accordance with the In-vitro Deposition Evaluation model.

4. The method of claim 1, wherein the oligomer comprises a soy oligomer.

5. The method of claim 1, wherein the oligomer is fully hydrogenated.

6. The personal cleansing composition of claim 1, wherein the oligomer is about 80% hydrogenated or more.

7. The personal cleansing composition of claim 1, wherein the benefit phase comprises from about 1% to about 13%, by weight of the benefit phase, of the oligomer.

8. The personal cleansing composition of claim 1, wherein the benefit phase comprises from about 2% to about 12%, by weight of the benefit phase, of the oligomer.

9. The personal cleansing composition of claim 1, wherein the benefit phase comprises from about 2% to about 10%, by weight of the benefit phase, of the oligomer.

10. The personal cleansing composition of claim 1, wherein the cleansing phase is structured.

11. The personal cleansing composition of claim 1, wherein the hydrophobic benefit agent comprises unsaturated soybean oil.

12. The personal cleansing composition of claim 1, wherein the hydrophobic benefit agent exhibits a Vaughan solubility parameter from about 5 to about 14 and exhibits a viscosity of about 1500 cP or less at from about 20° C. to about 25° C.

13. A method of enhancing the hydration of skin, comprising:
providing a rinse-off personal cleansing composition comprising a cleansing phase comprising a surfactant and water and a benefit phase comprising a benefit agent and from about 1% to about 15% of an oligomer derived from metathesis of unsaturated polyol esters, and
instructing an individual to apply the composition to the skin,
wherein the level of hydration is in accordance with at least one of the following: a Corneometer improvement of 0.1 Corneometer units or more as compared to the personal cleansing composition without the oligomer after treatment of at least once a day for at least 3 days when measured in accordance with the Corneometer Testing Method; a dry skin grade improvement of about 0.4 units or more at 3 hours after a treatment versus the personal cleansing composition without the oligomer when measured in accordance with the Dry Skin Grade Method; a TEWL improvement of about 0.5 grams of water per hour per m² or more after treatment at least once a day for 14 days when measured in accordance with the TEWL Method, or a combination thereof.

14. The method of claim 13, wherein the oligomer comprise a soy oligomer.

15. The method of claim 13, wherein the oligomer is fully hydrogenated.

16. The method of claim 13, wherein the oligomer is about 80% hydrogenated or more.

17. The method of claim 13, wherein the cleansing phase and the benefit phase are blended.

18. The method of claim 13, wherein the benefit phase comprises from about 2% to about 12%, by weight of the benefit phase, of the oligomer.

19. The rinse-off multi-phase personal cleansing composition of claim 13, wherein the benefit phase comprises from about 2% to about 10%, by weight of the benefit phase, of the oligomer.

20. The method of claim 1, wherein the Corneometer improvement is from about 0.2 Corneometer units to about 1.0 Corneometer units as compared to the personal cleansing composition without the oligomer after treatment of at least once a day for at least 3 days when measured in accordance with the Corneometer Testing Method; a dry skin grade improvement of about 0.4 units to about 2.0 units at 3 hours after a treatment versus the personal cleansing composition without the oligomer when measured in accordance with the Dry Skin Grade Method; a TEWL improvement of about 0.6 grams of water per hour per m² to about 5.0 grams of water per hour per m² after treatment at least once a day for 14 days when measured in accordance with the TEWL Method, or a combination thereof.
